(19) United States
(12) Patent Application Publication
(10) Pub. No.: US 2026/0341480 A1
(43) Pub. Date: Oct. 29, 2026

(54) SYSTEM AND METHOD FOR PERFORMING WORKFLOW AND PERFORMANCE ANALYSIS OF MEDICAL PROCEDURE

(71) Applicant: KONINKLIJKE PHILIPS N.V., EINDHOVEN (NL)

(72) Inventors: JIN LIU, MALDEN, MA (US); LUCAS DE MELO OLIVEIRA, WILMINGTON, MA (US); PURAN ADESH BHAGAT, ANDOVER, MA (US)

(21) Appl. No.: 19/103,741

(22) PCT Filed: Aug. 16, 2023
(86) PCT No.: PCT/EP2023/072564

§ 371 Date: Feb. 13, 2025

Related U.S. Application Data
(60) Provisional application No. 63/403,034, filed on Sep. 1, 2022.

(51) Int. Cl. G16H 40/20

(52) U.S. Cl. CPC G16H 40/20

(57) ABSTRACT

A system and method are provided for performing performance analysis of a type of procedure performed in a medical facility. The method includes providing a procedure log for the medical facility, which stores information logging steps of procedures; providing a predefined event list for the type of procedure; performing NLP on the procedure log to extract keywords respectively associated with the key events in the predefined event list; creating a final list of the extracted keywords respectively assoociated with the key events in the predefined event list; generating a process workflow for each of the procedures based on the final list of the extracted keywords and the procedure log formatted by a data interface; performing performance performance analysis of efficiency of performing the type of procedure in the medical facility by calculating time-related matrices and KPIs based on the process workflow; and displaying the performance analysis at a display.

### BACKGROUND

The healthcare industry is putting more emphasis on data and analysis with regard to operational efficiency, which generally requires healthcare data to be automatically processed in a scalable and robust manner. For example, hospitals include specialized medical facilities, such as interventional labs (e.g., catheterization labs (cath labs)), operating rooms (ORs), and combined labs, that are expensive to run. Many hospitals have shifted from “fee-for service” reimbursement model toward “quality-based reimbursement” by insurance providers, which increases pressure on the hospitals to reduce cost and utilize resources as efficiently as possible. Therefore, it is important to monitor and ultimately improve operational efficiency of the medical facilities.

Further, accreditation programs and regulations of hospitals typically require the medical facilities (e.g., interventional labs) to keep detailed procedure logs, which are documented by healthcare professionals (e.g., nurses) in real-time. Procedure logs, which may be referred to as event logs, are typically is part of nursing documentation. The procedure logs document detailed steps or events happening during procedures in the medical facilities and corresponding timestamps. Such events may include patient arrival, physician arrival, procedure start, and procedure stop, for example. With the detailed steps in the procedure logs, it is possible to retrieve very detailed workflow information for analyzing the efficiency of the procedures performed in the medical facilities.

However, it is presently difficult to accurately and timely access data points from the procedure logs that could provide insights and input for analytics pipelines, making it challenging to use such data points to obtain operational efficiency. For example, the healthcare professionals must manually search for information from procedure logs, as well as other relevant data, in several “siloed” data sources. This is very time-consuming, error prone, not scalable, and imposes an unnecessary burden on the healthcare professionals.

### SUMMARY

According to a representative embodiment, a method is provided for performing performance analysis of a type of procedure performed in a medical facility. The method includes providing a procedure log for the medical facility, where the procedure log stores information logging steps of procedures of the type of procedure performed in the medical facility; providing a predefined event list for the type of procedure performed in the medical facility, where the predefined event list includes key events for the type of the procedure; performing natural language processing (NLP) on the procedure log using an NLP algorithm to extract keywords respectively associated with the key events in the predefined event list; creating a final list of the extracted keywords respectively associated with the key events in the predefined event list; generating a process workflow for each of the procedures; performing performance analysis of efficiency of performing the type of procedure in the medical facility by calculating time-related matrices and key performance indicators (KPIs) based on the process workflow; and displaying the performance analysis at a display connected to a user interface.

According to another representative embodiment, a system is provided for performing performance analysis of a type of procedure performed in a medical facility. The system includes a user interface connected with a display; at least one processing unit; and at least one non-transitory memory. The at least one non-transitory memory stores a procedure log for the medical facility, where the procedure log stores information logging steps of procedures of the type of procedure performed in the medical facility, and a predefined event list for the type of procedure performed in the medical facility, where the predefined event list includes key events for the type of the procedure. The non-transitory memory further stores instructions that, when executed by the at least one processing unit, cause the at least one processing unit to perform NLP on the procedure log using an NLP algorithm to extract keywords respectively associated with the key events in the predefined event list; create a final list of the extracted keywords respectively associated with the key events in the predefined event list; generate a process workflow for each of the procedures; perform performance analysis of efficiency of performing the type of procedure in the medical facility by calculating time-related matrices and KPIs based on the process workflow; and display the performance analysis at the display.

According to another representative embodiment, a non-transitory computer readable medium is provided for storing instructions for performing performance analysis of a type of procedure performed in a medical facility. When executed by at least one processing unit, the instructions cause the at least one processing unit to perform NLP on a procedure log using an NLP algorithm for the medical to extract keywords respectively associated with key events included in a predefined event list for the type of the procedure, where the procedure log stores information logging steps of procedures of the type of procedure performed in the medical facility; create a final list of the extracted keywords respectively associated with the key events in the predefined event list; generate a process workflow for each of the procedures; perform performance analysis of efficiency of performing the type of procedure in the medical facility by calculating time-related matrices and KPIs based on the process workflow; and display the performance analysis at a display connected to a user interface.

### BRIEF DESCRIPTION OF THE DRAWINGS

The example embodiments are best understood from the following detailed description when read with the accompanying drawing figures. It is emphasized that the various features are not necessarily drawn to scale. In fact, the dimensions may be arbitrarily increased or decreased for clarity of discussion. Wherever applicable and practical, like reference numerals refer to like elements.

FIG. 1 is a simplified block diagram of a system for performing workflow and performance analysis of a medical facility, according to a representative embodiment.

FIG. 2 is a simplified block diagram of memory modules storing instructions for performing workflow and performance analysis of a medical facility, according to a representative embodiment.

FIG. 3 shows illustrative tables showing similarity scores of extracted keywords relative to a predefined event list, according to a representative embodiment.

FIG. 4 shows an illustrative workflow in table form generated by workflow generation module, according to a representative embodiment.

FIG. 5 shows an illustrative Gantt chart generated by a user interface, according to a representative embodiment.

FIG. 6 is a flow diagram of a method for performing workflow and performance analysis of a medical facility, according to a representative embodiment.

### DETAILED DESCRIPTION

In the following detailed description, for the purposes of explanation and not limitation, representative embodiments disclosing specific details are set forth in order to provide a thorough understanding of an embodiment according to the present teachings. Descriptions of known systems, devices, materials, methods of operation and methods of manufacture may be omitted so as to avoid obscuring the description of the representative embodiments. Nonetheless, systems, devices, materials and methods that are within the purview of one of ordinary skill in the art are within the scope of the present teachings and may be used in accordance with the representative embodiments. It is to be understood that the terminology used herein is for purposes of describing particular embodiments only and is not intended to be limiting. The defined terms are in addition to the technical and scientific meanings of the defined terms as commonly understood and accepted in the technical field of the present teachings.

It will be understood that, although the terms first, second, third, etc. may be used herein to describe various elements or components, these elements or components should not be limited by these terms. These terms are only used to distinguish one element or component from another element or component. Thus, a first element or component discussed below could be termed a second element or component without departing from the teachings of the inventive concept.

The terminology used herein is for purposes of describing particular embodiments only and is not intended to be limiting. As used in the specification and appended claims, the singular forms of terms “a,” “an” and “the” are intended to include both singular and plural forms, unless the context clearly dictates otherwise. Additionally, the terms “comprises,” “comprising,” and/or similar terms specify the presence of stated features, elements, and/or components, but do not preclude the presence or addition of one or more other features, elements, components, and/or groups thereof. As used herein, the term “and/or” includes any and all combinations of one or more of the associated listed items.

Unless otherwise noted, when an element or component is said to be “connected to,” “coupled to,” or “adjacent to” another element or component, it will be understood that the element or component can be directly connected or coupled to the other element or component, or intervening elements or components may be present. That is, these and similar terms encompass cases where one or more intermediate elements or components may be employed to connect two elements or components. However, when an element or component is said to be “directly connected” to another element or component, this encompasses only cases where the two elements or components are connected to each other without any intermediate or intervening elements or components.

The present disclosure, through one or more of its various aspects, embodiments and/or specific features or sub-components, is thus intended to bring out one or more of the advantages as specifically noted below. For purposes of explanation and not limitation, example embodiments disclosing specific details are set forth in order to provide a thorough understanding of an embodiment according to the present teachings. However, other embodiments consistent with the present disclosure that depart from specific details disclosed herein remain within the scope of the appended claims. Moreover, descriptions of well-known apparatuses and methods may be omitted so as to not obscure the description of the example embodiments. Such methods and apparatuses are within the scope of the present disclosure.

Generally, in order to understand and improve operations in various medical facilities, such as interventional labs, healthcare professionals need to have access to workflow information that describes events during procedures stored in an information system used in the medical facilities. According to various embodiments, procedure logs in the medical facilities are collected and processed in a scalable way by leveraging natural language processing (NLP) algorithms, and automatically generating workflow and operational efficiency analysis. Semantic similarity and a new term frequency methods are used to find customized keywords for workflow event identification and timestamp extraction.

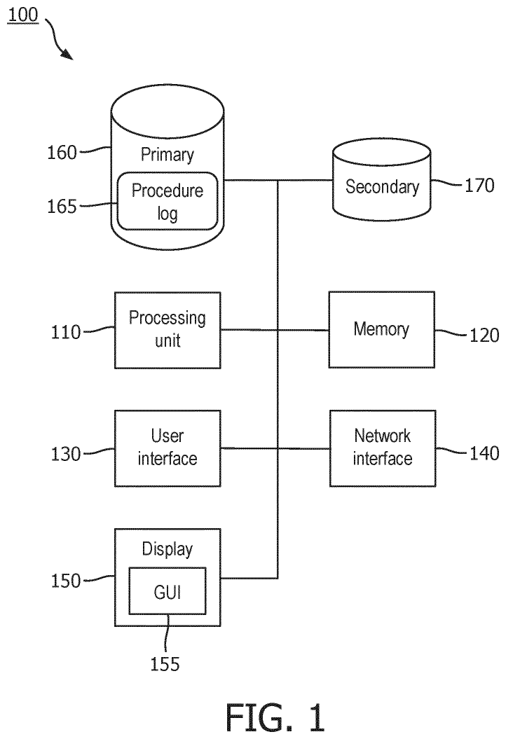

FIG. 1 is a simplified block diagram of a system for performing workflow and performance analysis of a medical facility in a hospital, according to a representative embodiment.

Referring to FIG. 1, system 100 includes a processing unit 110 and memory 120 for storing instructions executable by the processing unit 110 to implement processes described herein. In addition, the system 100 includes a user interface 130 for interfacing with a user, a network interface 140 for interfacing with other components and instruments, and a display 150, which may include graphical user interface (GUI) 155. The system 100 further includes or otherwise connects to primary data source 160 and secondary (optional) data source 170.

The processing unit 110 is representative of one or more processing devices, and is configured to execute software instructions to perform functions as described in the various embodiments herein. The processing unit 110 may be implemented by one or more servers, general purpose computers, central processing units, processors, microprocessors or microcontrollers, state machines, programmable logic devices, FPGAs, ASICs, or combinations thereof, using any combination of hardware, software, firmware, hard-wired logic circuits, or combinations thereof. As such, the term “processing unit” encompasses an electronic component able to execute a program or machine executable instructions, and may be interpreted to include more than one processor or processing core, as in a multi-core processor and/or parallel processors. The processing unit 110 may also incorporate a collection of processors within a single computer system or distributed among multiple computer systems, such as in a cloud-based or other multi-site application. Programs have software instructions performed by one or multiple processors that may be within the same computing device or which may be distributed across multiple computing devices.

The processing unit 110 may include an AI engine or module, which may be implemented as software that provides artificial intelligence, such as NLP algorithms, and may apply machine learning, such as artificial neural network (ANN), convolutional neural network (CNN), or recurrent neural network (RNN) modeling, for example. The AI engine may reside in any of various components in addition to or other than the processing unit 110, such as the memory 120, an external server, and/or the cloud, for example. When the AI engine is implemented in a cloud, such as at a data center, for example, the AI engine may be connected to the processing unit 110 via the internet using one or more wired and/or wireless connection(s), e.g., via the network interface 140.

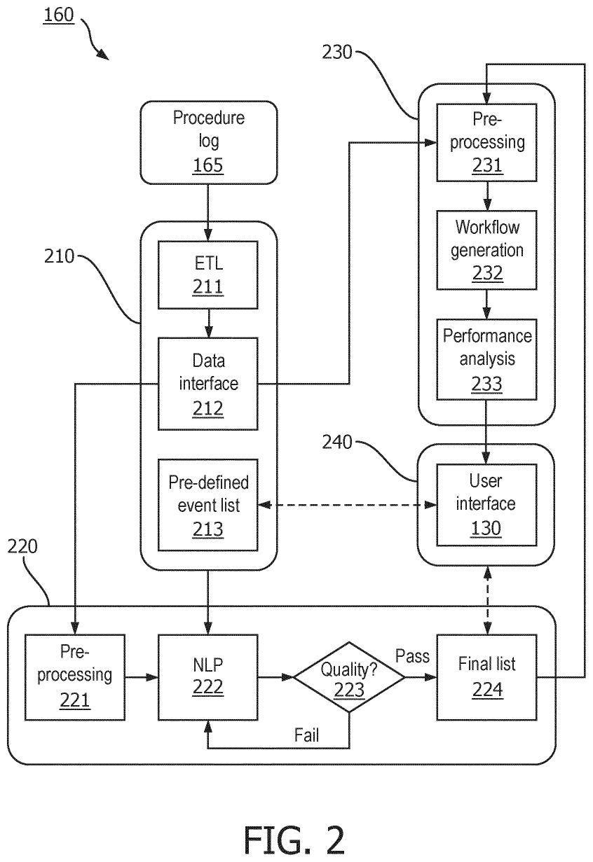

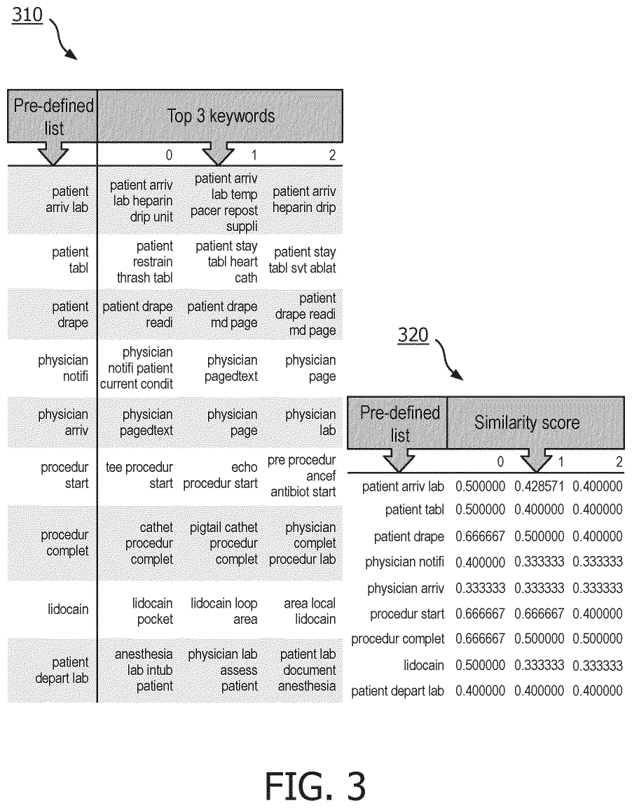

The memory 120 may include a main memory and/or a static memory, where such memories may communicate with each other and the processing unit 110 via one or more buses. The memory 120 stores instructions used to implement some or all aspects of methods and processes described herein, including the functions and methods described above with reference to FIGS. 2 and 3, for example. The memory 120 may include software modules, for example, as shown in FIG. 2. The memory 120 may be implemented by any number, type and combination of random access memory (RAM) and read-only memory (ROM), for example, and may store various types of information, such as software algorithms, data based models including ANNs, CNNs, RNNs, and other neural network based models, and computer programs, all of which are executable by the processing unit 110. The various types of ROM and RAM may include any number, type and combination of computer readable storage media, such as a disk drive, flash memory, an electrically programmable read-only memory (EPROM), an electrically erasable and programmable read only memory (EEPROM), registers, a hard disk, a removable disk, tape, compact disk read only memory (CD-ROM), digital versatile disk (DVD), floppy disk, blu-ray disk, a universal serial bus (USB) drive, or any other form of computer readable storage medium known in the art.

The memory 120 is a tangible storage medium for storing data and executable software instructions, and is non-transitory during the time software instructions are stored therein. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. A non-transitory storage medium is defined to be any medium that constitutes patentable subject matter under 35 U.S.C. § 101 and excludes any medium that does not constitute patentable subject matter under 35 U.S.C. § 101. The memory 120 may store software instructions and/or computer readable code that enable performance of various functions. The memory 120 may be secure and/or encrypted, or unsecure and/or unencrypted.

The user interface 130 provides information and data output by the processing unit 110 to the user and/or receives information and data input by the user. That is, the user interface 130 enables the user to enter data and to control or manipulate aspects of the processes described herein, and also enables the processing unit 110 to indicate the effects of the user's control or manipulation. All or a portion of the user interface 130 may be implemented by the GUI 155, viewable on the display 150. The user interface 130 may include a mouse, a keyboard, a trackball, a joystick, a haptic device, a touchpad, a touchscreen, and/or voice or gesture recognition captured by a microphone or video camera, for example, or any other peripheral or control to permit user feedback from and interaction with the processing unit 110. The display 150 may be a monitor such as a computer monitor, a television, a liquid crystal display (LCD), an organic light emitting diode (OLED), a flat panel display, a solid-state display, or a cathode ray tube (CRT) display, or an electronic whiteboard, for example.

The network interface 140 provides information and data output by the processing unit 110 to other components and/or instruments, e.g., that require one or more of the clock output signals. The network interface 140 may include one or more of ports, drives, or other types of interconnect and/or transceiver circuitry.

The primary data source 160 may include a hemodynamic recording system or a cardiovascular information system (CVIS), for example, and stores procedure log 165 for logging steps or events of procedures performed in the medical facility. The procedure log 165 includes both current and historical procedure log data, as further discussed below. The secondary data source 170 may include an electronic medical records (EMR) system and the CVIS, for example. The secondary data source 170 may be included to make the workflow and performance analysis more complete.

For purposes of explanation, the memory 120 is described as including modules, each of which includes the machine executable instructions (e.g., in software or computer programs) corresponding to an associated capability of the system 100. FIG. 2, in particular, is a simplified block diagram of modules in the memory 120 of FIG. 1 storing instructions for performing workflow and performance analysis of a medical procedure, according to a representative embodiment.

Referring to FIG. 2, the memory 120 includes first module 210 for data acquisition and preparation, second module 220 for natural language processing (NPL), third module 230 for workflow generation and performance analysis, and fourth module 240 for user interface (via the user interface 130 and the display 150).

The first module 210 includes extraction, transform and loading (ETL) module 211, data interface 212, and predefined event list 213. The ETL module 211 connects with hospital informatics systems, including the primary data source 160 (e.g., which includes the procedure log 165) and the secondary data source 170 (not shown in FIG. 2), to extract, transform and load relevant data records, including clinical, operational and demographic data into the data interface 212, discussed below. The ETL module 211 may interface with the primary data source 160 and optionally the secondary data source 170 using any compatible IT communication protocol, such as Health Level Seven (HL7), Digital Imaging and Communications in Medicine (DICOM), and Fast Health Interoperability Resources (FHIR), for example, as would be apparent to one skilled in the art.

The procedure log 165 is provided for the medical facility, such as an interventional lab (e.g., cath lab) or an operating room, for example, for which workflow and performance analysis are to be performed. The procedure log 165 includes information logged by medical personal, such as nurses, during each procedure performed in the medical facility. For example, the procedure log 165 may include logged information pertaining to heart catheterizations performed in the interventional lab. The information may include paired procedure description text and timestamps, case study date, case unique identification number (ID) and patient ID, for example.

As mentioned above, the procedure log 165 includes both historical procedure log data and current procedure log data. Generally, the current procedure log data refers to a single procedure (case) of interest. It does not necessarily mean a procedure that is currently being performed or that has been most recently performed. The current procedure log data is used in the third module 230 for analyzing real-time efficiency of procedures performed in the medical facility, as discussed below. The historical procedure log data refers to the procedure log data that has been collected from operation of the medical facility over a period of time. The historical procedure log data is used in the second module 220 for initializing configuration.

The secondary data source 170 is provided for the hospital (in which the medical facility is located), and includes more general information about each case. For example, the secondary data source 170 may include (i) case information from the CVIS and/or a hemodynamic recording system, such as study date, case type, participants, and case ID; (ii) case ordering information from the EMR system, such as care order time, scheduled time, case type (e.g. elective or primary), patient ID, and case study date; (iii) patient admission and discharge information from the EMR system, such as admission and discharge date, time and status, and patient ID; and (iv) physician report information from the EMR system (or the CVIS and/or hemodynamic recording system), such as description of procedures, diagnosis, physician summaries, case study date, case ID and patient ID. The secondary data source 170 helps to perform a more complete workflow and performance analysis, discuss below with reference to the third module 230. The patient and case records from different data sources may be linked together using common information, such as patient ID (e.g., medical record number), case ID, study date and/or admission information.

The data interface 212 is configured to receive the information extracted, transformed and loaded by the ETL module 211 from different data sources, including the primary and secondary data sources 160 and 170, and to format the procedure log 165 using the extracted information. The data interface 212 thus consolidates patient and case information, enabling easy access by the second and third modules 220 and 230. The formatted procedure log represents relationships between various elements within the data interface 212, as well as conditions to be preserved, and generally provides direction on how the data from the various sources are to be interpreted, as would be apparent to one skilled in the art. Timestamps from the procedure log 165 are also initially stored in the data interface 212 for creation of the workflow, discussed below.

The predefined event list 213 provides a list of key events based on the type of procedure for which the workflow and performance analysis is being performed. For example, the predefined event list 213 for an interventional lab used for interventional heart catheterizations may include as key events such as patient arrive lab, patient draped, physician notified, physician arrive, procedure start, lidocaine injection, procedure complete, and patient depart lab. Generally, the key events are the same as or otherwise correspond to entries in the procedure log 165. The predefined event list 213 may be created manually by an expert in the type of procedure to which it applies or by a learning algorithm, such as a ANN, CNN or RNN algorithm with machine learning, that is based on historic data collected from past procedures and corresponding performance analysis of the same type.

The second module 220 includes a preprocessing module 221, an NLP module 222, a quality check module 223, and a final list 224. Generally, the second module 220 finds customized text in historical procedure log data from the procedure log 165 associated with the key events in the predefined event list 213. The preprocessing module 221 receives the historical procedure log data of the procedure log 165 from the data interface 212 output from the first module 210, e.g., through a single procedure layer. The preprocessing module 221 performs text preprocessing on procedure description text in the historical procedure log data. The text preprocessing may include sentence segmentation and text normalization, as would be apparent to one skilled in the art. The sentence segmentation segments the text, which may be a combination of multiple sentences, into manageable segment sentences. The text normalization may include tokenization, removing stop-words, converting text to lowercase, stemming, and so on. The output of the preprocessing module 221 is a list of event sentences that is unique to the particular medical facility.

The NLP module 222 analyzes the event sentences in the list of event sentences to extract keywords. The NLP module 222 implements an NLP algorithm that performs text similarity analysis (or similarity scoring) on the event sentences to find the most similar text. The NLP algorithm cannot be performed in the human mind, and may be a learning algorithm, such as an ANN, CNN or RNN algorithm, with machine learning, for example. Based on this approach, the text similarity may be classified as string-based similarity, corpus-based similarity, knowledge-based similarity and/or hybrid similarity, as would be apparent to one skilled in the art. The string-based similarity shows only lexical similarity (surface closeness) and is relatively easy to calculate using metrics such as Jaccard similarity coefficient, Dice similarity coefficient and Cosine similarity, for example. The corpus-based similarity, the knowledge-based similarity and the hybrid similarity may be used to show semantic similarity (meaning closeness). The knowledge-based similarity, in particular, when using WordNet, may be further grouped as path-based, information content-based measures, feature-based, and hybrid.

The text similarity scoring may include converting the event sentences as vectors, which may be referred to as sentence embedding. The sentence embedding may be performed using a number of known approaches, such as term frequency-inverse document frequency (TF-IDF), word2vec technique and bidirectional encoder representations from transformers (BERT) language model, for example. With sentences converted as vectors, similarity scores (s_{1,ij}) between each unique event sentence (eᵢ) from the list of event sentences and each event text (dⱼ) in the predefined event list 213 are calculated to prioritize the event sentences that are semantically similar to the predefined event list 213. The computation of the similarity scores (S_{1,ij}) may be obtained using standard similarity scoring techniques, such as Jaccard similarity coefficient, Dice similarity coefficient, Cosine similarity, for example, as mentioned above, or other machine learning methods.
FIG. 3 shows illustrative tables showing similarity scores of extracted keywords relative to the predefined event list 213, according to a representative embodiment. Referring to FIG. 3, Table 310 includes a first column that lists terms associated with nine events from the predefined event list 213, and second through fourth columns that list the top three keywords that are similar to the terms in the first column, respectively, which may be extracted from the event sentences respectively. Table 320 includes a first column which lists the same terms as the first column in Table 310, and second through fourth columns that list similarity scores associated with the top three keywords in the second through fourth columns in Table 310, respectively. Generally, the more similar the keywords are to the corresponding terms from the predefined event list 213, the higher the similarity scores.

In addition to the similarity scoring of the event sentences, the NLP module 222 performs frequency scoring. The frequency scoring considers how frequently to obtain each event sentence at the case-level. The frequency of appearance of an event sentence (eᵢ) in the case may be calculated and normalized to obtain a frequency score (s_{2,ij}).

The frequency score may be performed, for example, using a variation of the TF-IDF method to make it suitable for procedure log event keyword extraction, according to a representative embodiment, although it is understood that the frequency score may be performed using other techniques without departing from the scope of the present teachings. That is, the frequency score (s_{2,ij}) is determined by Equation (1):

[symbol]

According to Equation (1), the frequency score (s_{2,ij}) is proportional to document frequency (DF) of the event sentence (eᵢ) and the inverse of term frequency difference (ITFD) of the event sentence (eᵢ) and the event text (dⱼ), where ITFD is the difference between the average number of times event sentence (eᵢ) appears in a case and the expected number of times event text (dⱼ) appears in the case. For example, when out of 100 cases, 93 cases have event sentence eᵢ in the corresponding procedure logs/documents, the document frequency of the event sentence eᵢ (DF(eᵢ)) is 0.93. ITFD also may be used jointly with the DF(eᵢ) to produce the frequency score (s_{2,ij}) when the expected term frequency is provided in the predefined event list 213. The term frequency (TF) is the number of times a term/sentence appears in a case in the procedure log 165. For example, in the predefined event list 213, event text (dⱼ) of “patient in the lab” is expected to happen only once for each case, so TF(dⱼ)=1. However, in the historical procedure log data of the procedure log 165, event sentence (eᵢ) of “patient arrives” may appear 1.02 times, as an example, in each case on average based on the previous year. ITFD may be calculated using a formula such as Equation (2), although ITFD may be calculated using other formulas without departing from the scope of the present teachings:

[symbol]

Calculation of ITFD results a number ranging from 0 to 1. In this case, the event sentence (eᵢ) is penalized when there is a large difference between the expected term frequency and the actual term frequency.

The NLP module 222 then performs keyword extraction based on the similarity and frequency scores. That is, the similarity and frequency scores may be combined to prioritize and finalize selection of keywords by applying a score threshold and/or selecting the top N event sentences. For example, the similarity and frequency scores may be combined through a weighted average as w₁*s₁+w₂*s₂, where w₁+w₂=1 and w₁, w₂>0.

After extraction of keywords, the quality check module 223 ascertains the quality of the keyword extraction process. To do so, the quality check module 223 analyzes the completeness and plausibility of finding the extracted keywords in each case record by checking the time sequence of events. Predetermined threshold(s) may be set to determine whether the similarity scoring and/or the frequency scoring need to be repeated. For example, a threshold for completeness of selected key events may be ⋅90%, and a threshold for plausibility of finding the extracted keywords may also be ⋅90%. When the quality of the key extraction process fails, the process returns to the NLP module 222, which repeats the keyword extraction based on the similarity and frequency scores. The NLP algorithm may be tuned, e.g., by the user or by known machine learning, prior to repeating the keyword extraction, such as increasing the threshold to reduce false positive, for example. When the quality of the key extraction process passes, the process proceeds to creating the final list 224.

The final list 224 is a list of keywords associated with each predefined event from the predefined event list 213, which is output from the second module 220. That is, the final list 224 includes keywords that were derived from the predefined event list 213 for events in the historical procedure log data of the procedure log 165 and extracted by the NLP module 222.

The final list 224 may be presented to the user through user interface 130 provided by the fourth module 240. This enables the user to confirm or provide feedback with regard to the list of keywords.

In an embodiment, the second module 220 only needs to be run once, for example, when the solution is deployed at a new medical facility (e.g., cath lab within a hospital) to initialize the configuration of keywords. The final list 224 of keywords may be confirmed by quality check and/or customer feedback. The functionality of the second module 220 need not be repeated, unless there is change in procedure log practice with regard to how information is logged in the procedure log 165.

The third module 230 includes a preprocessing module 231, a workflow generation module 232, and a performance analysis module 233. Generally, the third module 230 generates a workflow by applying the customized list of keywords in the final list 224 output from second module 220 in order to extract timestamps from the procedure log 165, and to analyze the operational performance of the medical facility (e.g., interventional lab) based on the extracted timestamps.

The preprocessing module 231 receives consolidated patient and case information, including the current procedure log data of the procedure log 165, from the data interface 212 output from the first module 210, e.g., through a single procedure layer. The preprocessing module 231 converts the current procedure log data into a standard or common format. For example, various terms for a physician, such as doctor, dr., physician and practitioner, may all be converted to “physician” for consistency. This may be done by applying the final list 224 of keywords provided by the second module 220 associated with the predefined events to the current procedure log, for example.

The workflow generation module 232 receives the preprocessed information from the preprocessing module 231 and the list of keywords from the final list 224 output from the second module 220. Each event in the current procedure log data from the procedure log 165 has an associated timestamp and persona (e.g., nurse, patient, cardiologist, assistant). The workflow generation module 232 extracts the timestamps, and associates the timestamps with the keywords in the final list 224 corresponding to these events. Each procedure performed in the medical facility therefore may be organized by event in chronological order using the extracted timestamps, enabling generation of a workflow for that procedure.

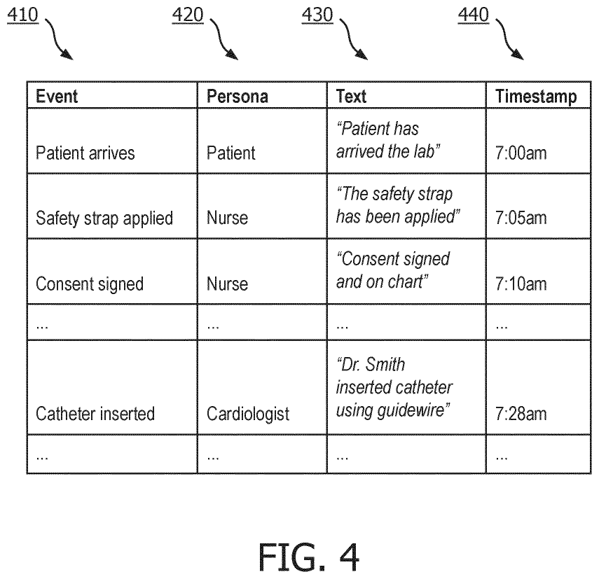

FIG. 4 shows an illustrative workflow in table form generated by the workflow generation module 232, according to a representative embodiment. Referring to FIG. 4, four illustrative events are shown taking place in an interventional lab during an interventional procedure. The workflow generation module 232 is able to build this workflow using the text in column 430 to identify the event in column 410. The order is determined using the timestamps in column 440 associated with the text (and thus the events). The persona listed in column 420 is also associated with the text.

In particular, the event “Patient Arrives” is identified by the text in the procedure log 165 “Patient has arrived at the lab,” which is timestamped 7:00 am. The event “Safety strap applied” is identified by the text in the procedure log 165 “The safety strap has been applied,” which is timestamped 7:05 am. The event “Consent Signed” is identified by the text in the procedure log 165 “Consent Signed and on chart,” which is timestamped 7:10 am. The event “Catheter Inserted” is identified by the text in the procedure log 165 “Dr. Smith inserted catheter using guidewire,” which is timestamped 7:28 am. The ellipses indicate the presence of additional events between and after those specifically depicted.

The performance analysis module 233 analyzes the workflow generated by the workflow generation module 232 to provide efficiency calculations for the medical facilities. The performance analysis may be performed by calculating a time-related matrix and key performance indicators (KPIs) based at least in part on the extracted timestamps of each case. For an interventional lab, for example, a time-related matrix may include information related to efficiency of the procedure, such as total case time (from patient arrival to patient departure), procedure time (from procedure start to procedure finish), patient preparation time in the interventional lab (from patient arrival to patient draped), physician delay (from physician notified to physician arrival), and first case start time. Turn-around time between cases for the interventional lab and/or the physician may be calculated by sorting cases according to the time and interventional lab and/or physician. The KPIs are indicators of the efficiency of the procedures, such as interventional lab utilization and lab on-time start ratio, time to start a procedure, how long a patient waits until a catheter is inserted, and how long to clean/prepare the room, for example. The interventional lab utilization is the ratio between the time during which the interventional lab is occupied and the time during which interventional lab is available. The lab on-time start ratio is the percentage of first scheduled cases that started before the targeted start time. The performance analysis module 233 outputs the efficiency calculations in a useful format to the fourth module 240.

The fourth module 240 includes the user interface 130 and the display 150 (not shown in FIG. 2). The user interface 130 and the display 150 together may provide a GUI for the user. The user interface 130 may be configured to take user feedback on customized keywords selected from the final list 224 output by the second module 220, indicated by the dashed arrow. The user interface 130 is also configured to visualize performance analysis results from the performance analysis module 233 output by the third module 230 on the display 150. The user interface 130 enables hospital facility managers to visualize the keyword selection from the processing of the procedure log 165, the workflow visualization and the facility operational performance.

With regard to user feedback collection for extracted keywords, the user is able to edit and/or confirm the extracted keywords from the final list 224 associated with events based on domain knowledge based on expertise.

With regard to the performance analysis visualization, the user interface 130 may build an efficiency dashboard and/or efficiency report using the workflow and the KPIs generated from third module 230 to show the operational efficiency in the medical facility. Also, filters may be applied for the user interface 130 to generate the efficiency report for a specific location and time range. Such filters may include hospital, medical facility, and time, for example, although other filters may be applied without departing from the scope of the present teachings. After filtering, results may be displayed automatically on the display 150, such as the number of cases performed during the time range and the corresponding KPIs, for example. In an embodiment, the KPIs may be color coded or otherwise highlighted to indicate levels of performance from good to poor. The efficiency dashboard shown on the display 150 may be directly linked (not shown) to the data interface 212 in the first module 210. This enables the data interface 212 to incorporate real-time results when new cases are being loaded and calculated.

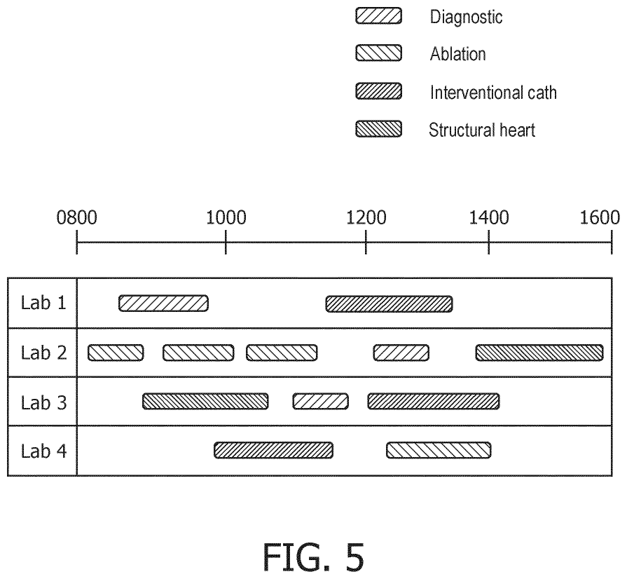

The user interface 130 and the display 150 may also provide overviews using any graphical representation for insight visualization using chart form for operational performance overview. Such graphical representation may include Gantt charts, for example, derived from the performance analysis module 233. FIG. 5 shows an illustrative Gantt chart generated by the user interface 130 and/or the performance analysis module 233 summarizing performance analysis, according to a representative embodiment.

Referring to FIG. 5, Gantt charts provide visual summaries to the user of how cases are distributed over four interventional labs (indicated as Lab 1, Lab 2, Lab 3, and Lab 4) over the course of an eight hour day, for example. Each bar represents the time from when a patient arrives in the medical facility to when the patient leaves the interventional lab for each procedure (case). The bars are color coded (shaded) to visually indicate the type of each procedure. The types of procedure for interventional labs may include a diagnostic procedure, an ablation procedure, an interventional heart catheterization procedure, or a structural heart procedure, for example. Of course, other types of charts, graphs, and visual displays may be incorporated without departing from the scope of the present teachings. For example, a bar plot may be used similarly to visualize total procedure time and a time series plot may be used to visualize how long patients wait until a physician arrives.

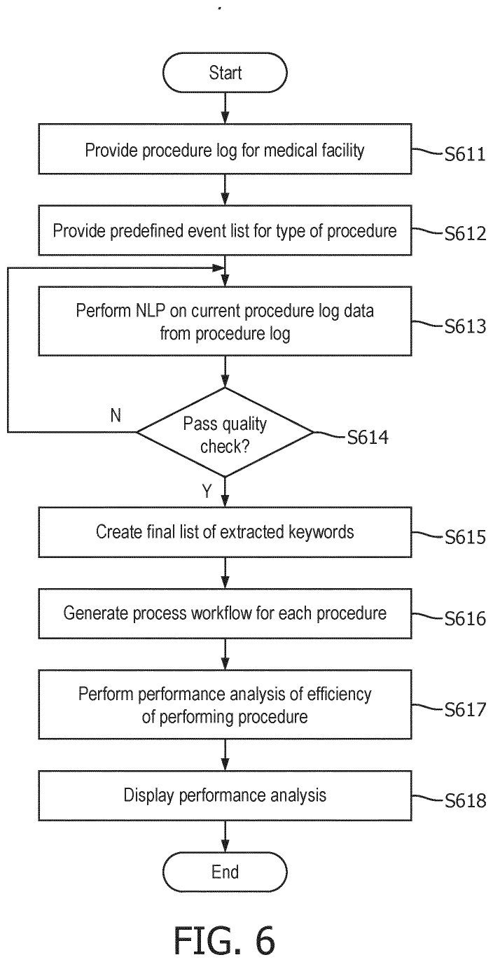

FIG. 6 is a flow diagram of a method for performing workflow and performance analysis of a medical facility, according to a representative embodiment. The method may be implemented by the system 100, discussed above, under control of the processing unit 110 executing instructions stored as the various software modules in the memory 120, for example.

Referring to FIG. 6, a procedure log (e.g., procedure log 165) is provided for a medical facility in block S611. The procedure log stores information logging steps of procedures of the type of procedure performed in the medical facility, and includes current procedure log data and historical procedure log data. Entries in the procedure log may be entered manually, e.g., by a nurse or other medical personal, or automatically, e.g., in response to triggers launched during the procedure in the medical facility, or a combination of manually and automatically. The procedure log may be formatted by a data interface (e.g., data interface 212), which is configured to transform the procedure log into an interface procedure log representation built from information extracted from various data sources.

In block S612, a predefined event list (e.g., predefined event list 213) is provided for the type of procedure performed in the medical facility. The predefined event list includes key events for the type of the procedure for which the workflow and performance analysis is being performed.

In block S613, NLP procedure (e.g., NLP module 222) is performed on historical procedure log data from the procedure log to extract keywords respectively associated with the key events in the predefined event list. Performing NLP may be preceded by text preprocessing of the historical procedure log data, including sentence segmentation and text normalization, for example.

In block S614, a quality check (e.g., quality check module 223) is performed on the extracted key words. The quality check includes analyzing completeness and plausibility by checking the time sequence of events. Respective predetermined thresholds may be set for completeness of selected key events and plausibility of finding the extracted keywords. When the extracted keywords fail the quality check (do not meet the predetermined thresholds), the process returns to block S613 to repeat performance the NLP on the current procedure log data. When the extracted keywords pass the quality check (meeting the predetermined thresholds), the process continues to block S615.

In block S615, a final list of the extracted keywords (e.g., final list 224) is created. The keywords in the final list are respectively associated with the key events in the predefined event list provided in block S612.

In block S616, a process workflow (e.g., workflow generation module 232) is generated for each of the procedures. The process workflow for each of the procedures may be generated by receiving the current procedure log data from the procedure log provided in block S611, receiving the final list of the extracted keywords created in block S615, extracting timestamps from the procedure log and associating the extracted timestamps with the extracted keywords from the final list, and ordering the key events chronologically using the extracted keywords and the associated timestamps. Generating the process workflow may be preceded by preprocessing of the current procedure log data, including converting the current procedure log data into a standard or common format, as discussed above.

In block S617, performance analysis of efficiency of performing the type of procedure in the medical facility (e.g., performance analysis module 233) is performed based on the process workflow. Performing the performance analysis may include calculating time-related matrices and key performance KPIs, for example.

In block S618, the performance analysis is displayed on a display at a user interface. The performance analysis have any format indicating efficiency factors of the performing the procedures in the medical facility, without departing from the scope of the present teachings.

In accordance with various embodiments of the present disclosure, the methods described herein may be implemented using a hardware computer system that executes software programs stored on non-transitory storage mediums. Further, in an exemplary, non-limited embodiment, implementations can include distributed processing, component/object distributed processing, and parallel processing. Virtual computer system processing may implement one or more of the methods or functionalities as described herein, and a processor described herein may be used to support a virtual processing environment.

The illustrations of the embodiments described herein are intended to provide a general understanding of the structure of the various embodiments. The illustrations are not intended to serve as a complete description of all of the elements and features of the disclosure described herein. Many other embodiments may be apparent to those of skill in the art upon reviewing the disclosure. Other embodiments may be utilized and derived from the disclosure, such that structural and logical substitutions and changes may be made without departing from the scope of the disclosure. Additionally, the illustrations are merely representational and may not be drawn to scale. Certain proportions within the illustrations may be exaggerated, while other proportions may be minimized. Accordingly, the disclosure and the figures are to be regarded as illustrative rather than restrictive.

One or more embodiments of the disclosure may be referred to herein, individually and/or collectively, by the term “invention” merely for convenience and without intending to voluntarily limit the scope of this application to any particular invention or inventive concept.

Moreover, although specific embodiments have been illustrated and described herein, it should be appreciated that any subsequent arrangement designed to achieve the same or similar purpose may be substituted for the specific embodiments shown. This disclosure is intended to cover any and all subsequent adaptations or variations of various embodiments. Combinations of the above embodiments, and other embodiments not specifically described herein, will be apparent to those of skill in the art upon reviewing the description.

The Abstract of the Disclosure is provided to comply with 37 C.F.R. § 1.72(b) and is submitted with the understanding that it will not be used to interpret or limit the scope or meaning of the claims. In addition, in the foregoing Detailed Description, various features may be grouped together or described in a single embodiment for the purpose of streamlining the disclosure. This disclosure is not to be interpreted as reflecting an intention that the claimed embodiments require more features than are expressly recited in each claim. Rather, as the following claims reflect, inventive subject matter may be directed to less than all of the features of any of the disclosed embodiments. Thus, the following claims are incorporated into the Detailed Description, with each claim standing on its own as defining separately claimed subject matter.

The preceding description of the disclosed embodiments is provided to enable any person skilled in the art to practice the concepts described in the present disclosure. As such, the above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents and shall not be restricted or limited by the foregoing detailed description.

1. A method of performing performance analysis of a type of procedure performed in a medical facility, the method comprising:
providing a procedure log for the medical facility, wherein the procedure log stores information logging steps of procedures of the type of procedure performed in the medical facility;
providing a predefined event list for the type of procedure performed in the medical facility, wherein the predefined event list includes key events for the type of procedure;
performing natural language processing (NLP) on the procedure log using an NLP algorithm to extract keywords respectively associated with the key events in the predefined event list;
creating a final list of the extracted keywords respectively associated with the key events in the predefined event list;
generating a process workflow for each of the procedures;
performing performance analysis of efficiency of performing the type of procedure in the medical facility by calculating time-related matrices and key performance indicators (KPIs) based on the process workflow; and
displaying the performance analysis at a display connected to a user interface.

2. The method of claim 1, wherein generating the process workflow for each of the procedures comprises:
receiving the final list of the extracted keywords;
receiving the procedure log formatted by a data interface using information extracted from a plurality of data sources;
extracting timestamps from the formatted procedure log, and associating the extracted timestamps with the extracted keywords from the final list; and
ordering the key events using the extracted keywords and the associated timestamps.

3. The method of claim 1, further comprising:
preprocessing description text of the information stored in the procedure log before performing the NLP, wherein the preprocessing includes sentence segmentation and text normalization to provide event sentences unique to the medical facility.

4. The method of claim 3, wherein performing the NLP on the procedure log comprises:
performing similarity scoring by converting the event sentences to vectors and calculating similarity scores between the vectors of the event sentences and event text in the predefined event list; and
performing frequency scoring by multiplying a document frequency (DF) of the event sentences and an inverse of a term frequency difference (ITFD) of the event sentences and the event text, wherein the ITFD is a difference between an average number of times the event sentences appear in a case and an expected number of times the event text appears in the case.

5. The method of claim 1, further comprising:
performing a quality check on the extracted key words following the NLP, wherein the quality check comprises analyzing completeness and plausibility of finding the extracted keywords.

6. The method of claim 1, wherein displaying the performance analysis comprises:
displaying a workflow visualization for each procedure of the type of procedure performed in the medical facility; and
displaying an operational performance overview visually indicating a length of time of each procedure.

7. The method of claim 6, wherein the operation performance overview is displayed as a Gantt chart.

8. The method of claim 1, wherein the medical facility comprises an interventional lab.

9. A system for performing performance analysis of a type of procedure performed in a medical facility, the system comprising:
a user interface connected with a display;
at least one processor;
at least one non-transitory memory storing:
a procedure log for the medical facility, wherein the procedure log stores information logging steps of procedures of the type of procedure performed in the medical facility;
a predefined event list for the type of procedure performed in the medical facility, wherein the predefined event list includes key events for the type of procedure; and
instructions that, when executed by the at least one processor, cause the at least one processor to:
perform natural language processing (NLP) on the procedure log using an NLP algorithm to extract keywords respectively associated with the key events in the predefined event list;
create a final list of the extracted keywords respectively associated with the key events in the predefined event list;
generate a process workflow for each of the procedures;
perform performance analysis of efficiency of performing the type of procedure in the medical facility by calculating time-related matrices and key performance indicators (KPIs) based on the process workflow; and
display the performance analysis at the display.

10. The system of claim 9, wherein the instructions cause the at least one processor to generate the process workflow for each of the procedures by:
receiving the final list of the extracted keywords;
receiving the procedure log formatted by a data interface using information extracted from a plurality of data sources;
extracting timestamps from the formatted procedure log, and associating the extracted timestamps with the extracted keywords from the final list; and
ordering the key events using the extracted keywords and the associated timestamps.

11. The system of claim 9, wherein the instructions further cause the at least one processor to:
preprocess description text of the information stored in the procedure log before performing the NLP, wherein the preprocessing includes sentence segmentation and text normalization to provide event sentences unique to the medical facility.

12. The system of claim 11, wherein the instructions cause the at least one processor to perform the NLP on the procedure log by:
performing similarity scoring by converting the event sentences to vectors and calculating similarity scores between the vectors of the event sentences and event text in the predefined event list; and
performing frequency scoring by multiplying a document frequency (DF) of the event sentences and an inverse of a term frequency difference (ITFD) of the event sentences and the event text, wherein the ITFD is a difference between an average number of times the event sentences appear in a case and an expected number of times the event text appears in the case.

13. The system of claim 9, wherein the instructions further cause the at least one processor to:
perform a quality check on the extracted key words following the NLP, wherein the quality check comprises analyzing completeness and plausibility of finding the extracted keywords.

14. The system of claim 9, wherein the instructions cause the at least one processor to display the performance analysis by:
displaying a workflow visualization for each procedure of the type of procedure performed in the medical facility; and
displaying an operational performance overview visually indicating a length of time of each procedure.

15. The system of claim 14, wherein the operation performance overview is displayed as a Gantt chart.

16. A non-transitory computer readable medium storing instructions for performing performance analysis of a type of procedure performed in a medical facility that, when executed by at least one processor, cause the at least one processing unit-processor to:
perform natural language processing (NLP) on a procedure log using an NLP algorithm for the medical facility to extract keywords respectively associated with key events included in a predefined event list for the type of procedure, wherein the procedure log stores information logging steps of procedures of the type of procedure performed in the medical facility;
create a final list of the extracted keywords respectively associated with the key events in the predefined event list;
generate a process workflow for each of the procedures;
perform performance analysis of efficiency of performing the type of procedure in the medical facility by calculating time-related matrices and key performance indicators (KPIs) based on the process workflow; and
display the performance analysis at a display connected to a user interface.

17. The non-transitory computer readable medium of claim 16, wherein the instructions cause the at least one processing unit processor to generate the process workflow for each of the procedures by:
receiving the final list of the extracted keywords;
receiving the procedure log formatted by a data interface using information extracted from a plurality of data sources;
extracting timestamps from the formatted procedure log, and associating the extracted timestamps with the extracted keywords from the final list; and
ordering the key events using the extracted keywords and the associated timestamps.

18. The non-transitory computer readable medium of claim 16, wherein the instructions further cause the at least one processor to:
preprocess description text of the information stored in the procedure log before performing the NLP, wherein the preprocessing includes sentence segmentation and text normalization to provide event sentences unique to the medical facility.

19. The non-transitory computer readable medium of claim 18, wherein the instructions cause the at least one processor to perform the NLP on the procedure log by:
performing similarity scoring by converting the event sentences to vectors and calculating similarity scores between the vectors of the event sentences and event text in the predefined event list; and
performing frequency scoring by multiplying a document frequency (DF) of the event sentences and an inverse of a term frequency difference (ITFD) of the event sentences and the event text, wherein the ITFD is a difference between an average number of times the event sentences appear in a case and an expected number of times the event text appears in the case.

20. The non-transitory computer readable medium of claim 16, wherein the instructions further cause the at least one processor to:
perform a quality check on the extracted key words following the NLP, wherein the quality check comprises analyzing completeness and plausibility of finding the extracted keywords.
